(19) United States
(12) Patent Application Publication
(10) Pub. No.: US 2020/0367890 A1
(43) Pub. Date: Nov. 26, 2020

(54) ANASTOMOSIS DEVICES

(71) Applicants: SURG SOLUTIONS, S.L., Barcelona (ES); HOSPITAL CLÍNIC DE BARCELONA, Barcelona (ES)

(72) Inventors: Jordi DESCARREGA ROMÁN, Barcelona (ES); German HEBER BELLEMMI, Viladecans (ES); Joan Xavier FONTDEVILA FONT, Barcelona (ES)

(73) Assignees: Surg Solutions, S.L., Barcelona (ES); Hospital Clínic de Barcelona, Barcelona (ES)

(21) Appl. No.: 16/634,118

(22) PCT Filed: Jul. 25, 2018
(86) PCT No.: PCT/EP2018/070185

§ 371 Date: Jan. 24, 2020

(30) Foreign Application Priority Data
Jul. 25, 2017 (EP) 17382490.5

(51) Int. Cl. A61B 17/11

(52) U.S. Cl. CPC A61B 17/11; A61B 2017/00867

(57) ABSTRACT

An anastomosis device having: a tubular body with a longitudinal axis, the body having: a longitudinal gap along the entire length of the tubular body, defining matching longitudinal edges on the tubular body, the tubular body being expandable in radial direction between a contracted configuration in which the tubular body is rolled up on itself around the longitudinal axis and has at least a first radial dimension, with one longitudinal edge rolled up inside the other, and an expanded configuration in which the tubular body has a substantially cylinder shape with a second radial dimension that is larger than the first radial dimension and with the longitudinal edges facing each other; and first and second end portions each intended to contact the inner surface of a lumen when the tubular body is in expanded configuration. Also provided are a method of manufacturing of an anastomosis device and a holding system.

The present disclosure relates to anastomosis devices and methods.

### BACKGROUND

In surgical procedures it is often necessary to perform vascular anastomosis, for example joining blood vessels end-to-end.

Anastomosis is traditionally done by manual surgical suture. However, manual suture is technically complex, and therefore it has a long learning curve, a significant degree of failures and a reliability that varies from one surgeon to another. Furthermore, it is a slow operation, which adds to ischemia time and to the overall time of the surgical procedure.

A number of devices have been developed for simplifying and increasing the reliability of manual suture, for example, there are known stent-like devices which are introduced into the vessels in a radially compressed position, and then expanded so they contact the vascular endothelium and maintain the vessel open.

Other known devices include two interlocking sleeves, each of them provided with projections to engage one of the vessels.

In an instance, it may have been that a medical apparatus for joining body lumens in a living body may include a puncture member having a lumen, a distal opening portion and a needle tip at the distal end of the lumen. The needle tip can puncture a tube wall of the body lumen to form a puncture site. The medical apparatus may include a tubular member in the lumen of the puncture member that expands radially outward and contracts radially inward and a plunger movable relative to the puncture member to release the tubular member from the distal opening portion of the puncture member. The tubular member may expand to be fixed to the puncture site when the tubular member is released from the distal opening portion of the puncture member.

In other instances, there may have been methods and systems provided for tissue stabilization. Some aspects may include a carrier member having a length, width, and thickness, wherein the length and width may be each at least two times greater than the thickness; attachment members extending from the carrier member and that engage connective tissue; and stabilizing members, each of which can couple a respective attachment member to the carrier member and is substantially positionally fixed relative to the carrier member.

However, such devices may not provide satisfactory solutions in all cases, and especially in the case of an anastomosis performed in microsurgery procedures, such as microvascular free flap surgery, which is where manual suture is more complex.

Another possible device may have two tubular parts joined through an intermediate portion or coil which may be longitudinally extended or contracted, which at least partially solves the above problems.

However, it has now been found that it is possible to further improve the device, especially in relation with its manufacture and handling.

### SUMMARY

The purpose of the present disclosure is providing anastomosis devices that solve at least partly the above disadvantages, intended to be used in microsurgical procedures and also, in general, surgery of vascular anastomosis.

In a first aspect, an anastomosis device is provided. The device includes:
a tubular body with a longitudinal axis, said body having:
a longitudinal gap along the entire length of the tubular body, defining matching longitudinal edges on the tubular body,
whereby the tubular body is expandable in radial direction between a contracted configuration in which the tubular body is rolled up on itself around the longitudinal axis and has at least a first radial dimension, with one longitudinal edge rolled up inside the other, and an expanded configuration in which the tubular body has a substantially cylinder shape with a second radial dimension that is larger than the first radial dimension and with the longitudinal edges facing each other; and
first and second end portions each intended to contact the inner surface of lumens of two vessels to be joined when the tubular body is in expanded configuration.

The tubular body in the contracted configuration may not have a generally cylindrical configuration so the body may have several radial dimensions in the radial direction. By the “first radial dimension” is meant at least one of these radial dimensions.

Thanks to that first aspect, the tubular body may be void of linkages, arms, coils, loops or the like so it may allow simpler manufacturing process and easier handling of the device than the previous devices. The tubular body may be more simply handled by the user than prior devices.

This configuration may also mean a robust and strong device which is less prone to suffer from fractures or the like.

As there are no linkages, arms, coils, or the like in the device, the risk of leakage may be negligible, particularly at the anastomosis site where the vessels are joined. Therefore, a better seal of the device may be obtained when it expands in a radial direction.

The anastomosis procedure may be simplified, because it requires less skill and less time, with all the advantages that this brings about. In certain circumstances it may allow completely avoiding the need for manual suture, because the blood vessels are held together by the device and become joined without the need of suture.

Furthermore, the configuration of the device is such that it can have a small size, suitable in microsurgery applications.

In some examples of the device, the longitudinal gap may be a rectilinear continuous gap from end to end of the tubular body. Alternatively, the longitudinal gap may have other shapes defined by matching longitudinal edges of the tubular body.

In further examples of the device, the tubular body may be a unitary rolled-up sheet. By unitary sheet is meant a sheet made from a single piece of material.

In further examples of the device, the first and second end portions have a different second radial dimension from each other. This way, an end-to-end anastomosis may be performed when one vessel may have a different diameter from another vessel. The diameter may progressively vary from one end portion to another end portion, so a laminar fluid along the tubular body may be promoted.

According to another aspect, a method of manufacturing an anastomosis device as herein disclosed is provided. The method includes:
forming a tubular body;
activating the tubular body to cause a contraction of said tubular body in radial direction by rolling up the tubular body on itself around its longitudinal axis.

Thus, an anastomosis device as herein described may be obtained in an easy manner.

In some examples, the method includes before forming a tubular body:
forming projections on an outer surface of first and second tubular end portions of the tubular body by engraving the tubular body in a planar configuration.

This way, the size of the anastomosis device may be even reduced since the projections may be easily engraved when the tubular body is in a planar configuration. Subsequently the tubular body may be formed with the projections.

According to another aspect, provided may be a holding system for handling an anastomosis device according to any of herein disclosed examples. The holding system includes:
a clamping device having a pair of cooperating jaws, wherein each jaw includes a recess and the recesses are complementary to each other so as to define an opening in a closed configuration of the clamping device, wherein the opening is configured to match, at least partially, the cross section of the anastomosis device in a contracted configuration.

Thanks to this aspect, the user can easily handle and keep the anastomosis device in the contracted configuration even when carrying out an anastomosis.

Throughout the present description, a “substantially cylinder shape” should be understood as including a generally round, elliptical or at least partially rounded cross-section. The expression “substantially cylinder shape” will also mean a frusto-conical shape.

### BRIEF DESCRIPTION OF THE DRAWINGS

Non-limiting examples of the present disclosure will be described in the following, with reference to the appended drawings, in which:

FIG. 1 schematically shows a view in perspective of an anastomosis device according to one example, in an activated position;

FIG. 2 schematically shows a view in perspective of the anastomosis device of FIG. 1 in an inactivated position;

FIG. 3 schematically shows a side view of the anastomosis device of FIG. 2;

FIG. 4 schematically shows a cross section view of the anastomosis device of FIG. 2;

FIG. 5 schematically shows the anastomosis device of FIG. 1 entering the lumens of vessels in an end-to-end anastomosis;

FIG. 6 schematically shows the anastomosis device of FIG. 2 inside the lumens of vessels in an end-to-end anastomosis;

FIG. 7 schematically shows a perspective view of a clamping device of a holding system according to an example;

FIG. 8 schematically shows a side view of the clamping device of FIG. 7;

FIG. 9 schematically shows a detail A of FIG. 8;

FIG. 10 schematically shows a side view of a clamping device according to a further example;

FIGS. 11A-C schematically shows a sequence of a radial expansion of an anastomosis device with the clamping device of FIG. 10;

FIG. 12 schematically shows a side view of a holding cap according to an example; and

FIG. 13 schematically shows a plan view of the holding cap of FIG. 12.

### DETAILED DESCRIPTION OF EXAMPLES

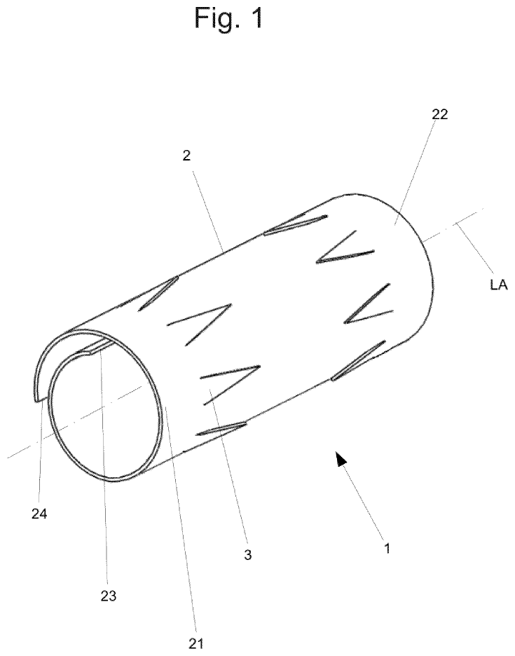

Some examples of an anastomosis device 1 as disclosed herein are shown in FIGS. 1-6. An example of anastomosis device 1 may include: a tubular body 2 with a longitudinal axis LA. Said body 2 may include: a longitudinal gap along the entire length of the tubular body 2, the longitudinal gap may define matching longitudinal edges 23, 24 on the tubular body 2. This way, the tubular body may be expandable in a radial direction RD between a contracted configuration in which the tubular body 2 may be rolled up on itself around the longitudinal axis LA and may have at least a first radial dimension, with one longitudinal edge rolled up inside the other, and an expanded configuration in which the tubular body 2 may have a substantially cylinder shape with a second radial dimension that may be larger than the first radial dimension and with the two longitudinal edges 23, 24 facing each other.

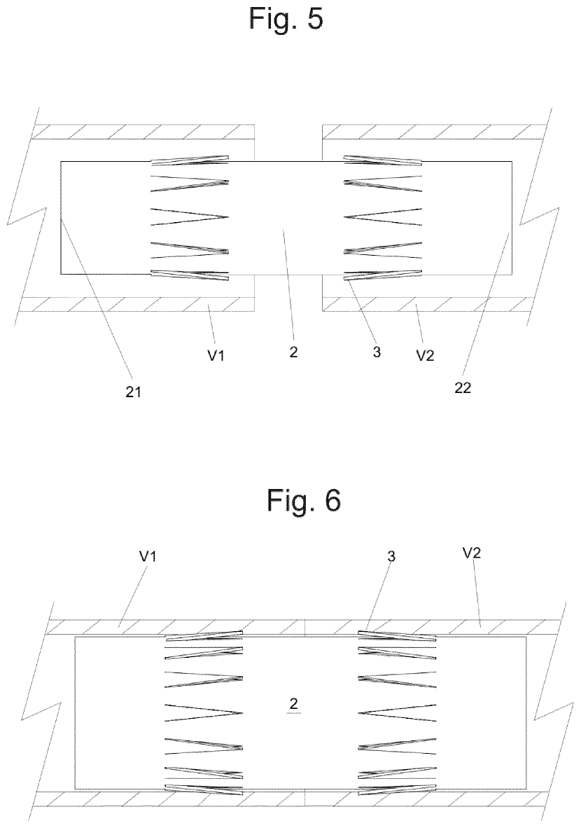

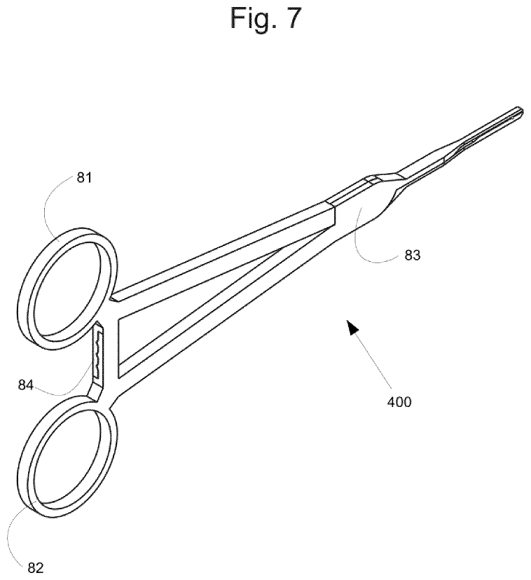

The tubular body 2 may also include first and second end portions 21, 22: each end portion may be intended to contact the inner surface of a lumen when the tubular body 2 is in expanded configuration as shown in FIGS. 5 and 6.

In some examples, the first radial dimension may be substantially in the range of 30%-50% the second radial dimension.

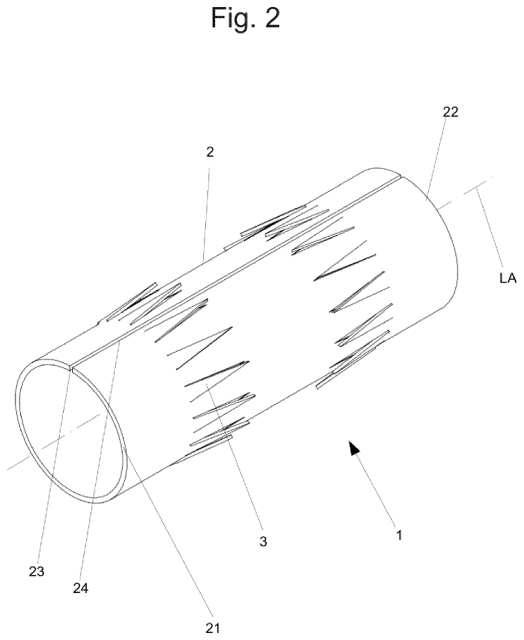

In the present disclosure, the whole tubular body 2 may be uniformly rolled up and rolled out as can be seen respectively in FIGS. 1 and 2. Both first and second tubular end portions 21, 22 may be integrally formed in a unitary body.

FIG. 1 schematically shows a view in perspective of an anastomosis device 1 according to one example, in an activated position (contracted configuration). It can be seen that the tubular body 2 is rolled up on itself about its longitudinal axis LA. The tubular body 2 may be radially compressed by rolling up one longitudinal edge inside another longitudinal edge. This way, the tubular body 2 may be kept contracted in a radial direction RD.

FIG. 2 schematically shows a view in perspective of FIG. 1 but in an inactivated position. The tubular body 2 may be rolled out about its longitudinal axis LA from the rolled-up position of the activated position. This way, the tubular body 2 may be kept in expanded configuration in the radial direction RD (as visible in FIG. 4). This inactivated position is shown in FIGS. 2, 3 and 4.

When the device 1 is in the inactivated position the longitudinal edges 23, 24 may be facing each other so as to form a device 1 with a generally cylindrical shape. This cylindrical shape is illustrated by FIG. 4.

As the tubular body 2 in the expanded configuration may have a generally cylindrical shape, it may have mainly one radial dimension such as the second radial dimension.

In FIGS. 2 and 4, the gap between the longitudinal edges 23, 24 has been oversized for the sake of clarity. However, this gap may be of negligible dimensions and both longitudinal edges 23, 24 may be in full or partial contact with each other.

The radial direction RD is substantially perpendicular to the longitudinal axis LA of the tubular body 2.

According to some examples, the tubular body 2 may include a rolled-up sheet. The sheet may be a laminar configuration element to be rolled up and be rolled out.

In some examples of the device 1 as that illustrated in the attached FIGS., the longitudinal gap may be a rectilinear continuous gap from end to end of the tubular body 2. In alternative examples, the gap may adopt different configurations which may be defined by the relative shape of the longitudinal edges 23, 24, such as a sawtooth or stepped shape.

That rectilinear gap may be in the longitudinal direction (taking into account the longitudinal axis LA) or slanted at a predetermined angle with respect to the longitudinal direction.

The device with the features disclosed in the present application does not break easily, and does not undergo a permanent, non-recoverable deformation when its shape is changed to the degree needed for its operation as described above.

In some examples, the tubular body 2 may include a shape-memory material. The tubular body 2 may be manufactured from that shape-memory material at least partially, for instance the tubular body 2 may include a region made of that kind of material.

Alternatively, the tubular body 2 may be made entirely from a shape-memory material.

The shape-memory material may be such that the tubular body 2 could be compressed to the contracted configuration in at least one radial direction RD at a temperature lower than the corporal temperature, and could recover the expanded configuration in said at least one radial direction RD, upon heating to the corporal temperature.

In some cases, especially in devices 1 of particularly small size such as those intended for microsurgery, the heat of the body may be sufficient for the tubular body 2 having shape-memory material to recover its initial shape (expanded configuration).

In other cases, it is foreseen to apply a source of external heating such as a heating fluid, in order to induce the shape recovery.

The shape-memory material may be for instance a shape-memory alloy or a shape-memory polymer.

The shape-memory alloy may be preferably a Nitinol alloy.

The shape-memory material may include preferably a PLLA polymer.

According to some examples, the anastomosis device 1 may be made of absorbable materials.

In some examples, the anastomosis device 1 may be made of superelastic materials.

It may be foreseen to provide an anastomosis device 1 such as disclosed that may also be drug-eluting, e.g. coated with a suitable substance such that after the procedure it may deliver the substance at and around the anastomosis site.

In FIGS. 1-6, it can be seen that the tubular body 2, and more specifically the first and second tubular end portions 21, 22, may include projections 3 on at least part of their outer surface which may be suitable for contacting, engaging or otherwise increasing the friction with endothelial tissue in the inner surface of the lumen of a blood vessel. In FIG. 1 only some projections 3 have been illustrated for the sake of clarity but projections 3 may be positioned around the outer face at least of first and second tubular end portions 21, 22 following a substantially circular path of the tubular body 2.

The projections 3 may be for instance spikes, surface roughness or the like.

In some examples, the projections 3 on the tubular end portions 21, 22 may be spike-shaped and inclined with respect to the longitudinal axis LA of the tubular body 2.

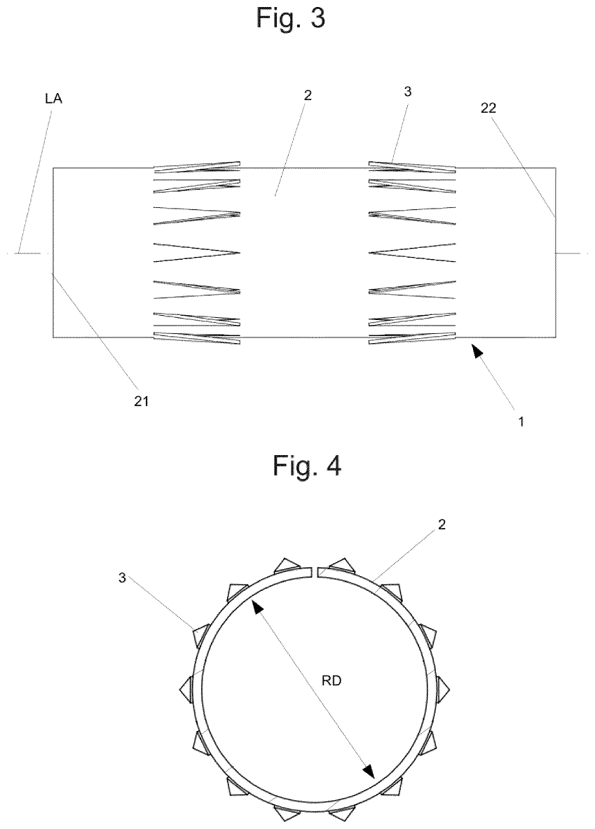

In some implementations, projections 3 on each of the first and second tubular end portions 21, 22 of the tubular body 2 may be inclined in opposite directions with respect to the longitudinal axis of the tubular body 2. The directions may be suitable to promote insertion of each of the first and second tubular end portions 21, 22 into the ends of blood vessels to be joined (see for instance FIGS. 5 and 6) and to oppose its withdrawal. These implementations may be show, for instance, in FIG. 3 where it is schematically illustrated a side view of the anastomosis device 1 according to examples herein disclosed.

In this manner, the projections 3 do not hinder the insertion of the ends of the tubular body 2 into the ends of two blood vessels or other lumens, and after insertion they provide very good attachment to pull the two vessels towards each other.

In the following, an example of use of one implementation of the anastomosis device 1 related to an end-to-end vascular anastomosis procedure will be explained.

The device 1 may be initially in the activated position (contracted configuration) as described above and illustrated in FIG. 1. This activated position may be ready to use since the tubular body 2 may be radially contracted and thus it may have a smaller first radial dimension relative the second radial dimension of the radially expanded tubular body of the FIG. 2. One longitudinal edge 23 of the exemplary sheet may be rolled up inside another longitudinal edge 24 of the sheet as per FIG. 1.

Then, two blood vessels to be joined may be brought sliding respectively on each of the end portions 21, 22 as illustrated in FIG. 5. Thanks to the configuration of the tubular body 2, one end portion 21 may be inserted into the lumen of one vessel V1 and the other end portion 22 may be inserted into the lumen of the other vessel V2. The size of the device 1 may be selected depending on the dimensions of the lumen of the vessel; for instance, the size of the device 1 may be such that the radial dimensions of the tubular body 2 in the activated, contracted position may be substantially smaller than the diameter of the lumen of the vessels.

According to one example, the tubular body 2 may have a length of about 3.5 mm and a diameter in an inactivated position of about 1.18 mm; the sheet of the tubular body 2 may have a width of about 0.045 mm. These dimensions could vary depending on the case.

The length of the tubular body 2 may be substantially the same in both the activated and inactivated position.

When both vessels are placed around the respective end portions 21, 22, the entire tubular body 2 configured as a single body may start to radially expand upon heating caused, for instance, by the blood temperature. In the case where the tubular body 2 is a unitary sheet, the rolled-up sheet may start expanding in the radial direction RD. As the sheet rolls out, the radial dimension of the tubular body 2 increases in at least one radial direction RD.

Because of the dimensions of the device 1 and the lumens of the vessels, as soon as the end portions 21, 22 are inserted into the respective lumens, the tubular body 2 may contact and engage the endothelial wall of the lumens through the projections 3.

At the end of the radial expansion of the tubular body 2, the device 1 is in the inactivated position as disclosed herein and the vessels to be joined may be maintained in complete contact with each other at the anastomosis site. FIG. 6 schematically shows the anastomosis device 1 in the inactivated position and inside the lumens of vessels V1, V2 in a vascular end-to-end anastomosis.

Good sealing may be obtained through the example of the device 1 of FIG. 2 owing to the pairs of longitudinal edges 23, 24 which may substantially abut each other in the inactivated position.

According to another example not shown, the longitudinal edges 23, 24 may be provided with a relative matching shape so as to provide an improved sealing effect.

According to another implementation not shown the device 1 may be coated for instance with a biocompatible polymeric material so as to obtain an improved fluid-tight sealing in the above mentioned gap between the longitudinal edges 23, 24.

In the inactivated, expanded condition of the device 1 illustrated in FIG. 2 the gap or clearance between the longitudinal edges 23, 24 is so narrow that there is substantially no risk of leakage.

The two vessels are thereafter maintained in position by the device 1, and this results in anastomosis of the vessels. This may occur without the need of any suture at all, or with a much smaller amount of suture with respect to manual suture anastomosis methods.

Thus, it will be appreciated from the above description that implementations of a device 1 as disclosed herein may allow multiple advantages such as fast anastomosis procedures, which do not require a high degree of skill from the surgeon, and also higher safety and reliability in the procedure, with a reduction of the risks of leaks, thrombosis, etc.

Furthermore, the device may be small in size and therefore suitable also for microsurgery applications, wherein manual suture is particularly complex. It may also be simple in construction and therefore relatively low-cost with respect to known devices such as couplers or those having several parts, springs, etc.

The present disclosure also relates to a method of manufacturing an anastomosis device 1 according to the herein disclosed examples. The method of manufacturing may include:
forming a tubular body 2;
activating the tubular body 2 to cause a contraction of said tubular body 2 in radial direction RD by rolling up the tubular body 2 on itself around its longitudinal axis LA.

In some examples, the tubular body 2 may be formed from a unitary sheet.

The method of manufacturing may further include: forming projections 3 on an outer surface of first and second tubular end portions 21, 22 of the tubular body 2 by engraving the tubular body 2 in a planar configuration. Those projections 3 may be engraved before forming the tubular body 2.

Those projections 3 may be engraved for example by laser cutting an exemplary unitary sheet in a planar configuration before forming the tubular body 2.

According to one example, the method of manufacturing may further include: making the tubular body 2 of a shape-memory material, and cooling the tubular body 2 at a temperature lower than corporal temperature when activating the tubular body 2.

After cooling the tubular body 2, it may be kept at a temperature lower than corporal temperature until it may be used.

According to another aspect, a holding system for handling an anastomosis device 1 according to any of the herein examples is provided. The holding system includes: a clamping device 400, 500 including a pair of cooperating jaws 401, 402, 501, 502, wherein each jaw 401, 402, 501, 502 includes a recess 405, 406, 505, 506 and the recesses 405, 406, 505, 506 are complementary to each other so as to define an opening 407, 507 in a closed configuration of the clamping device. The opening 407, 507 is configured to match, at least partially, the cross section of the anastomosis device 1 in a contracted configuration.

Therefore, the opening 407, 507 of the clamping device may be configured to surround, at least partially, the perimeter of the tubular body 2 of the anastomosis device 1 when the device is in the activated configuration.

Parts of the clamping system could be made from any suitable medical grade materials.

According to some examples the holding system may be used with anastomosis devices 1 made from either memory-shape or superelastic materials.

In some examples, the jaws 401, 402, 501, 502 may completely surround the perimeter of the tubular body in the activated configuration.

In some examples, the opening 407, 507 may hold the cross section of the tubular body 2 along 12.5% of its overall length at most. Thanks to this feature the ends of the vessels V1, V2 to be anastomosed may be brought together more closely while the user may handle the tubular body 2.

In some examples, the opening 407, 507 may have a substantially rounded cross section.

In some examples, the clamp 400, 500 may be configured so as to keep the body 2 in activated configuration, wherein the first radial dimension may be about 30% lower than the second radial dimension.

By closed configuration of the clamping device is meant a configuration wherein the tips of the jaws 401, 402, 501, 502 are meeting or are about to meet. When the tips of the jaws are meeting or are about to meet they can exert a significant force on a body or the like placed between them. The closed configuration would be the opposite to an open configuration wherein the jaws are more relatively separated and cannot exert substantially the significant or even any force on a body or the like placed between them.

The recess 405, 406, 505, 506 may be disposed substantially in a perpendicular direction to the length of the clamp 400, 500. This way the anastomosis device may be also positioned substantially in a perpendicular direction to the length of the clamp 400, 500.

In some examples, it may be envisaged that the recesses 405, 406, 505, 506 are provided with a surface coating or finishing so as to enhance the friction between the clamp 400, 500 and the tubular body 2.

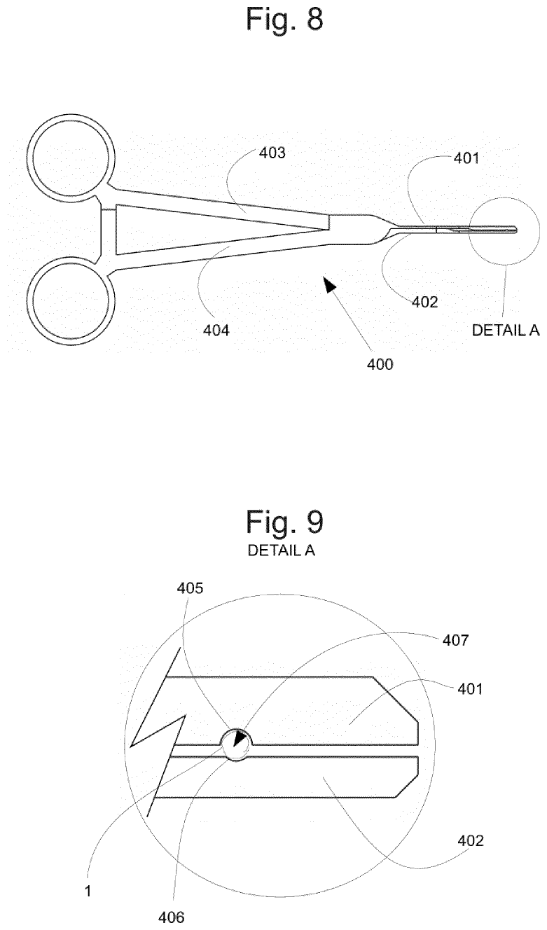

FIG. 7 schematically shows a perspective view of a clamping device 400 of a holding system according to an example and FIG. 8 schematically shows a side view of the clamping device of FIG. 7. FIG. 9 schematically shows a detail A of FIG. 8.

Some examples of the clamp 400 may include two jaws 401, 402, shanks 403, 404, finger bows 81, 82 or the like, a joint or fulcrum 83 for relative movement of jaws and a ratchet mechanism 84, see for instance FIGS. 7 or 8.

In order to hold a portion of the tubular body 2 along the opening 407, the user may place that portion of the body 2 in a recess 405 or 406 when the clamp 400 is in the open configuration. The tubular body 2 may be in the contracted or activated configuration. Then the user may close the jaws 401, 402 to hold a portion of the tubular body 2 between them. Afterwards, the user may perform an anastomosis as herein disclosed.

The user may hold the tubular body 2 through the opening 407 at any point along the length of the body 2.

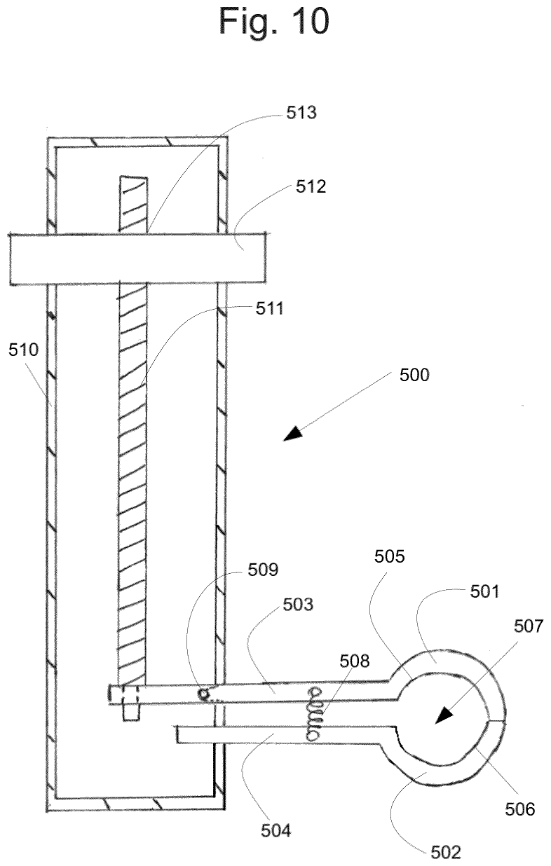

FIG. 10 schematically shows a side view of a clamping device 500 according to a further example. In FIG. 10 some parts have illustrated in section view for the sake of clarity.

According to a further example of the holding system, the clamping device 500 may further include:
a handle 510 to which the jaws 501, 502 may be detachably attached;
wherein the handle 510 may include a jaw regulating mechanism 511, 512 configured to adjust the relative distance between the jaws 501, 502.

In some examples, the handle 510 may include an elongated body configured to be grasped by a hand of the user.

As can be seen in the example of FIG. 10, each jaw 501, 502 may include an arm 503, 504 which is joined to a curved portion where the recesses 505, 506 are positioned. When the recesses 505, 506 are brought together to form the opening 507, the curved portions may substantially form a ring.

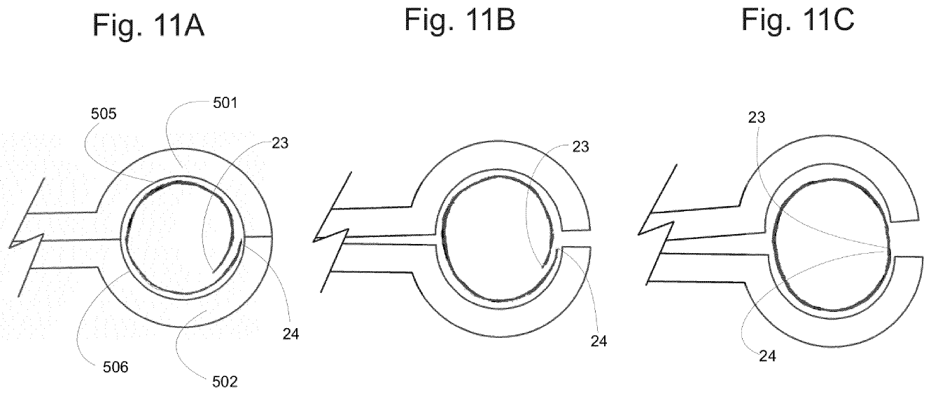

In the example illustrated in FIGS. 10 and 11, the curved portions of the jaws 501, 502 are “U”-shaped.

In some examples, one jaw 501 may be pivotally attached to the handle 510 and the jaw regulating mechanism 511, 512 may include a shaft 511 associated with the jaw 501 in such a way that a linear movement of the shaft 511 in axial direction may cause the jaw 501 to tilt with respect to the handle 510. Alternatively, jaw 502 could be pivotally attached to the handle 510.

In some examples, the jaw 504 may be detachably attached to the shaft 511.

In some further examples, both jaws 501, 502 could be pivotally attached to the handle 510.

The pivoting jaw 501 may be additionally attached to the handle 510 by a pivot joint 509. The shaft 511 may be attached to the pivoting jaw 501 substantially in an end opposite to the recess 505 and the pivot joint 509 may be placed at an arm 503 between the recess 505 and the shaft 511. Thus, a linear motion in axial direction of the shaft 511 may cause the jaw 501 to tilt.

In some examples, the shaft 511 may be threaded and the jaw regulating mechanism may further include a wheel 512 which may include a threaded through hole 513, wherein the shaft 511 may be longitudinally disposed across the through hole 513 in such a way that the shaft 511 meshes the through hole 513. A rotation of the wheel 512 about the length of handle 510 may cause the linear motion of the shaft 511 in axial direction of the handle 510. The wheel 512 may be fixed in a position along the length of the handle so as to cause the linear motion of the shaft 511 with a rotation of the wheel 512.

Alternatively, the linear motion of the shaft may be achieved by an electric actuator or the like.

In some examples, the clamping device 500 may include an elastic element 508 such as a spring, recoil or the like, configured to oppose the relative separation of jaws 501, 502. In the example of FIG. 10, the elastic element 508 is a spring fixed to each jaw 501, 502.

In some examples, the handle 510 and the jaws 501, 502 may form a clamping device 500 with a generally “L” shaped configuration. The “L” shape may provide the user with an enhanced visibility of the tubular body 2 and thus a better control on the radial expansion of thereof.

In some examples, the jaws 501, 502 may be disposable. The elastic element 508 may be disposable as well.

An anastomosis device in activated configuration may be placed in the opening 507 and the jaws 501, 502 may exert a tension over the cross section of the body 2. The tension may be created by the elastic element 508. The tubular body 2 may be kept in a contracted configuration in spite of its superelastic characteristics because of the jaws 501, 502 may surround the cross section of the body 2 by the recesses 505, 506.

Then, the user may attach the jaws 501, 502 with the anastomosis device 1 to the clamping device 500. The pivot joint 509 may be disposed in the pivoting jaw 501 and the handle 510 may include a notch to receive the pivot joint 509.

A set of jaws keeping the anastomosis device 1 in activated configuration may be delivered to be detachably attached to the handle 510.

By actuating the regulating mechanism, the user may relatively separate the jaws 501, 502 from each other. In the example of FIG. 10, by turning the wheel 512, the meshed shaft 511 may be moved towards the jaws 501, 502. Then, an end of the pivoting jaw 501 may be pushed in the same direction. The pivoting jaw 501 may tilt around the pivoting axis 509 so as to separate the recesses 505, 506 from each other. This separation may reduce the tension over the cross section of the body 2. Therefore, the tubular body 2 may start to radially expand.

FIGS. 11A-C schematically shows a sequence of a radial expansion of an anastomosis device with the clamping device of FIG. 10.

The user may gradually expand the body 2 when the end portions 21, 22 are already placed inside vessels V1 and V2. By turning further the wheel 512 the body may be further expanded. This way, the user may control the expansion of the anastomosis device 1 from the contracted configuration of FIG. 1 to the expanded configuration of FIG. 2. FIG. 11 provide an exemplary sequence of expansion. The sequence has been illustrated with three FIGS., although the radial expansion may be performed through a different number of steps.

For actuating the wheel, a user may grasp the handle 510 with one hand and rotate the wheel 512 with the other hand. The rotation axis of the wheel 512 may be parallel to the length of the handle 510.

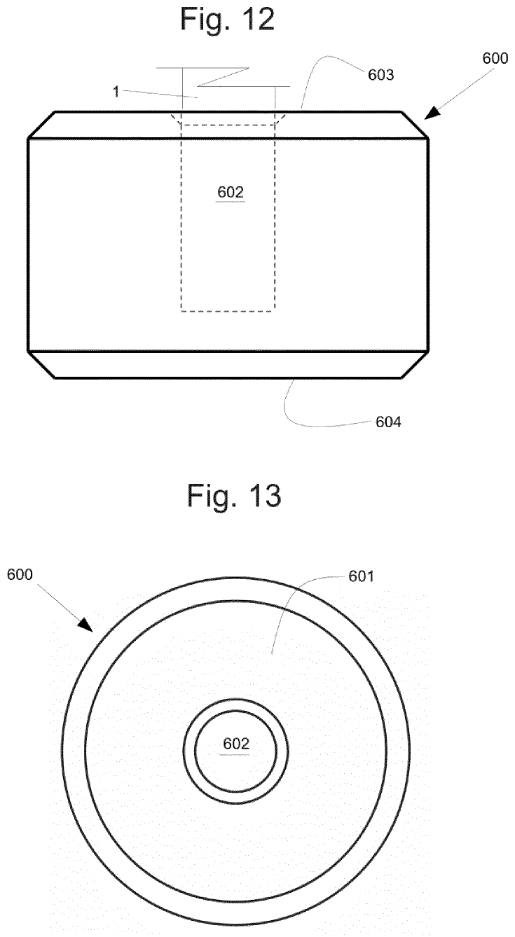

FIG. 12 schematically shows a side view of a holding cap according to an example; and FIG. 13 schematically shows a plant view of the holding cap of FIG. 12.

In some examples, the holding system may further include a holding cap 600, wherein the cap 600 may include a cavity 602 to receive at least a portion of the cross section of the anastomosis device 1, wherein the cavity 602 may be configured to match at least partially the cross section of the anastomosis device 1 in a contracted configuration. One of the first and second end portions 21, 22 may be received by the cavity 602.

The holding cap 600 may include a cap body 601 having a top and bottom faces 603, 604. In FIG. 12, the mouth of the cavity 602 is placed in the top face 603 but it could be placed in the bottom face 604.

The cavity 602 may have a shape to match the cross section of the tubular body in a contracted configuration. In FIG. 12, it can be seen that the cavity 602 may have a generally rounded cross section.

In some examples, the cavity may be adapted to hold a longitudinal part of one end portion 21, 22 for instance from the very beginning until the projections 3 which may be positioned around the outer face of the tubular body 2.

In some example, the clamping system further includes two holding caps 600, one cap 600 for each end portion 21, 22.

The anastomosis device 1 may be delivered with at least one of its end portions 21, 22 received in the cavity of the holding cap 600. The user may grip the tubular body 2 by or through the clamping device 400, so as to release the tubular body 2 from the holding cap 600. In the case of the clamping device 400, the tubular body 2 would be received in the opening 407. Then the tubular body 2 may be used to perform an anastomosis of two vessels V1, V2 as herein disclosed.

The features of the holding cap 600 herein disclosed could not be necessarily combined with the features of the rest of parts of holding systems described herein. For instance, a holding system could include the holding cap 600 without the rest of the herein disclosed examples.

The present disclosure also relates to a method for end-to-end vascular anastomosis. Such a method may include:
providing an anastomosis device 1 as disclosed in any of the herein disclosed examples;
activating the device 1, i.e. causing the tubular body 2 to be radially contracted in radial direction RD. The device 1 may achieve a configuration as per FIG. 1;
inserting the first and a second tubular end portions 21, 22 respectively in a first and second blood vessels to be anastomosed to each other; and
inactivating the device, i.e. allowing the tubular body 2 to radially expand in said radial direction RD. The device 1 may achieve a configuration as per FIG. 2.

After a first degree of expansion of the tubular end portions 21, 22 of the single tubular body 2, at least part of the outer surface of these tubular end portions 21, 22 engages and becomes attached to the endothelial tissue of the first and second vessels V1, V2 (as described above). See for instance, FIGS. 5 and 6.

A holding system according to any of the herein disclosed examples may be used while performing the anastomosis. The clamping devices 400, 500 may keep the anastomosis device 1 in a contracted configuration and the user may handle the device 1 to insert the first and second tubular end portions 21, 22 in the first and second vessels V1, V2.

According to an example, jaws 401, 402, 501, 502 may hold the tubular body 2 at about a middle way from the first and second end portions 21, 22. Both end portions 21, 22 may protrude from the jaws 401, 402, 501, 502 in a substantially perpendicular direction to the length of the jaws. That is to say, the longitudinal axis LA of the device 1 may be positioned substantially perpendicular to the length of the jaws when the user handles the anastomosis device 1.

In the examples where the anastomosis device 1 is handled with the clamping device 500, the user may control the radial expansion of the anastomosis device 1 as aforementioned. The user may decide the amount and the duration of the expansion. The radial expansion may be carried out in a gradual manner.

Several options are foreseen for the activation of the device, i.e. for applying a force to radially compress the tubular body 2, and also for the inactivation of the device 1, i.e. for allowing or causing the expansion of the tubular body 2.

For example, the device may be activated immediately before it has to be employed in a vascular anastomosis procedure.

Alternatively the activation may be performed as a step at the end of the manufacturing process, and the device may then be packaged and stored in activated position until it is used. It may be achieved by either the holding cap 600 or the clamps 501, 502.

If it is activated immediately before use, the activation may be performed manually by the surgeon or other medical practitioner, or by using a suitable tool.

In some examples, the activation of the device 1 may be performed at a temperature lower than the corporal temperature.

If the material of the tubular body is a shape memory material, the activation of the device 1 may be performed for example keeping the tubular body 2 at a temperature lower than about 25° C.

If the activation is done upon manufacture, the device may be thereafter handled and stored in suitable positions, e.g. suitable temperature positions, to avoid its inactivation before use.

The inactivation of the device after the tubular body has been inserted in the corresponding lumens may occur when the corporal temperature heats the device 1 and the shape memory material recovers the initial shape. However, it is also possible to increase or decrease the speed of inactivation by contacting the device 1 with hot or cold serum, or a similar heat or cold source.

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow. If reference signs related to drawings are placed in parentheses in a claim, they are solely for attempting to increase the intelligibility of the claim, and shall not be construed as limiting the scope of the claim.

1. An anastomosis device comprising:
a tubular body with a longitudinal axis, said body comprising:
a longitudinal gap along the entire length of the tubular body, defining matching longitudinal edges on the tubular body,
the tubular body being expandable in a radial direction between a contracted configuration in which the tubular body is rolled up on itself around the longitudinal axis and has at least a first radial dimension, with one longitudinal edge rolled up inside the other, and an expanded configuration in which the tubular body has a substantially cylinder shape with a second radial dimension that is larger than the first radial dimension and with the longitudinal edges facing each other; and
first and second end portions each configured to contact the inner surface of lumens of two vessels to be joined when the tubular body is in expanded configuration.

2. The device according to claim 1, the longitudinal gap being a rectilinear continuous gap from end to end of the tubular body.

3. The device according to claim 1, the tubular body being a unitary rolled-up sheet.

4. The device according to claim 1, the tubular body comprising a shape-memory material.

5. The device according to claim 4, the shape-memory material and the tubular body being configured to be compressed to the contracted configuration at a temperature lower than the corporal temperature, and to recover the expanded configuration upon heating to the corporal temperature.

6. The device according to claim 4, the shape-memory material being one or more of an alloy, a Nitinol alloy, or a shape-memory polymer.

7. The device according to any of claims 1, the first and second tubular end portions comprising projections on at least part of their outer surface, configured for engaging an inner surface of the lumen.

8. The device according to claim 7, the projections on the tubular end portions being spike-shaped and inclined with respect to the longitudinal axis of the tubular body.

9. The device according to claim 8, the projections on each of the first and second tubular end portions being inclined in opposite directions with respect to the longitudinal axis of the tubular body.

10. The device according to claim 1, the longitudinal edges substantially abutting each other in an inactivated position.

11. The device according to claim 1, the first and second end portions having a different second radial dimension from each other.

12. A method for end-to-end vascular anastomosis comprising:
providing an anastomosis device that comprises:
a tubular body with a longitudinal axis, said body comprising:
a longitudinal gap along the entire length of the tubular body, defining matching longitudinal edges on the tubular body,
the tubular body being expandable in a radial direction between a contracted configuration in which the tubular body is rolled up on itself around the longitudinal axis and has at least a first radial dimension, with one longitudinal edge rolled up inside the other, and an expanded configuration in which the tubular body has a substantially cylinder shape with a second radial dimension that is larger than the first radial dimension and with the longitudinal edges facing each other; and
first and second end portions each configured to contact the inner surface of lumens of two vessels to be joined when the tubular body is in expanded configuration;
the method for end-to-end vascular anastomosis further comprising:
activating the device to cause the tubular body to be radially contracted in radial direction;
inserting the first and a second tubular end portions respectively in a first and a second blood vessel to be anastomosed to each other; and
inactivating the device, the tubular body radially expanding in said radial direction.

13. The method according to claim 12, further comprising:
forming projections on an outer surface of first and second tubular end portions of the tubular body by engraving the tubular body in a planar configuration.

14. The method according to claim 12, further comprising:
cooling the tubular body at a temperature lower than corporal temperature when activating the tubular body, the tubular body being made of shape-memory material.

15. The method according to claim 12, the activating the device comprising rolling up the tubular body on itself around its longitudinal axis.

16. A holding system for handling an anastomosis device that comprises:
a tubular body with a longitudinal axis, said body comprising:
a longitudinal gap along the entire length of the tubular body, defining matching longitudinal edges on the tubular body,
the tubular body being expandable in a radial direction between a contracted configuration in which the tubular body is rolled up on itself around the longitudinal axis and has at least a first radial dimension, with one longitudinal edge rolled up inside the other, and an expanded configuration in which the tubular body has a substantially cylinder shape with a second radial dimension that is larger than the first radial dimension and with the longitudinal edges facing each other; and
first and second end portions each configured to contact the inner surface of lumens of two vessels to be joined when the tubular body is in expanded configuration;
the holding system comprising:
a clamping device comprising a pair of cooperating jaws, each jaw comprising a recess and the recesses are complementary to each other so as to define an opening in a closed configuration of the clamping device, the opening being configured to match, at least partially, the cross section of the anastomosis device in a contracted configuration.

17. The holding system according to claim 16, the clamping device further comprising:
a handle to which the jaws are detachably attached;
the handle comprising a jaw regulating mechanism configured to adjust the relative distance between the jaws.

18. The holding system according to claim 17, one jaw is being pivotally attached to the handle and the regulating mechanism comprising a shaft associated with the jaw in such a way that a linear movement of the shaft in axial direction causes the jaw to tilt with respect to the handle.

19. The holding system according to claim 18, the shaft being threaded and the jaw regulating mechanism further comprising a wheel which comprises a threaded through hole, the shaft being longitudinally disposed across the through hole in such a way that the shaft meshes the through hole.

20. The holding system according to claim 16, further comprising a holding cap, the holding cap comprising a cavity to receive at least a portion of the cross section of the anastomosis device, the cavity being configured to match at least partially the cross section of the anastomosis device in the contracted configuration.
